(19) United States
(12) Patent Application Publication
(10) Pub. No.: US 2024/0393231 A1
(43) Pub. Date: Nov. 28, 2024

(54) SAMPLE HOLDER FOR SUPPORTING A HAIR STRAND, HAIR STRAND ANALYSIS KIT AND METHOD

(71) Applicant: L'OREAL, Paris (FR)

(72) Inventors: Benjamin ASKENAZI, Saint Ouen (FR); Tal MARCU, Clichy (FR); Thibault CHAUFFIER, Clichy (FR); Thierry WASSERMAN, Clichy (FR)

(73) Assignee: L'OREAL, Paris (FR)

(21) Appl. No.: 18/686,129

(22) PCT Filed: Sep. 1, 2022
(86) PCT No.: PCT/EP2022/074354

§ 371 Date: Feb. 23, 2024

(30) Foreign Application Priority Data
Sep. 3, 2021 (FR) FR2109234

(51) Int. Cl. G01N 21/01; A61B 5/00; G01N 33/483

(52) U.S. Cl. CPC G01N 21/01; A61B 5/448; A61B 5/6838; G01N 33/4833

(57) ABSTRACT

The sample holder (12) for supporting a hair strand comprises at least a first arm (24), the first arm (24) comprising an inner face (28) and an outer face (30), the sample holder (12) defining a housing (32) for receiving the hair strand delimited at least in part by the first arm (24).

The first arm (24) comprises a through orifice arranged facing the housing (32) for receiving the hair strand and intended to removably receive an optic (18) of a hair strand analysis device (16), the first arm (24) comprising attachment means intended to removably attach the optic (18) of the analysis device (16) to the outer face (30) of the first arm (24).

The present invention relates to a sample holder for supporting a hair strand, the sample holder comprising at least a first arm, the first arm comprising an inner face and an outer face, the sample holder defining a housing for receiving the hair strand delimited at least in part by the first arm.

In the light of the great diversity of cosmetic products, and particularly hair products, present on the market, there is a growing desire among consumers to use personalized hair products, particularly adapted to the condition and color of their hair and their scalp.

Hair products are for example, shampoos, conditioners, treatments, colors, etc.

To determine the hair products to be applied according to the individuals, it is known, for example from the document U.S. Pat. No. 9,316,580, to analyze various parameters of a hair strand using a measuring device, for example using an optical scanner.

In order to increase the reliability of these measurements, it is necessary to select a representative hair strand comprising a sufficient amount of hair and, furthermore, to position the hairs of the strand in a satisfactory manner. More specifically, this requires stretching the hairs so that they are aligned in parallel with one another.

Generally, the hair strand is selected between the fingers and is stretched by pulling. However, such a selection and such a positioning in a manual manner are unsatisfactory due to the lack of precision thereof.

It is also known to use a sample holder, such as a hair clamp for example, mounted on the scanner of the analysis device and thus making it possible to clamp a hair strand according to a desired position.

However, such a clamp is not completely satisfactory.

Indeed, it is difficult to position such a clamp, as the measurement device scanner limits the possibility of moving the clamp and can thus impede the correct positioning thereof.

One aim of the invention is that of providing a sample holder capable of supporting a hair strand intended to be analyzed, that is easy to position and positioning the hair strand in a satisfactory manner.

For this purpose, the invention relates to a sample holder of the type cited above, characterized in that the first arm comprises a through orifice arranged facing the housing for receiving the hair strand and intended to removably receive an optic of a hair strand analysis device, the first arm comprising attachment means intended to removably attach the optic of the analysis device to the outer face of the first arm.

Thanks to the removable attachment of the optic of the analysis device, the sample holder can be positioned without the optic being attached to the sample holder, which facilitates the positioning thereof thus ensuring precise positioning of a hair strand to be analyzed.

In a specific embodiment of the invention, the sample holder comprises a second arm, each arm comprising an inner face and an outer face, the inner faces of the two arms defining therebetween the housing for receiving the hair strand.

The sample holder is a clamp, the first arm and the second arm being hinged on one another, the inner faces of the two arms being capable of coming against one another.

Such a sample holder comprising two arms makes it possible to clamp the hair strand between the two arms, which makes it easier to hold the hair strand to be analyzed in position.

In a specific embodiment of the invention, the inner face of the second arm defines a groove delimiting the housing for receiving the hair strand supported.

The through orifice of the first arm is defined facing the groove.

The groove comprises a bottom, the bottom being a monochrome surface, a transparent surface, a reflective surface or including patterns.

Such a groove makes it possible to delimit the size of the housing for receiving the hair strand, and thus limit the width and thickness of the hair strand to be analyzed to the desired dimensions while keeping the hairs aligned and stretched. The through orifice thus defines a capture opening facing the hairs held in the groove. The optic of the analysis device can thus make measurements on the hairs stretched and aligned in the groove, which increases measurement precision.

In a specific embodiment of the invention, the second arm comprises bulges defined near the groove.

The groove comprises combing teeth.

Such features facilitate the insertion and positioning of the hairs in the groove.

In a specific embodiment of the invention, the outer face of the first arm comprises a housing for accommodating the optic of the analysis device arranged around the through orifice.

The accommodating housing is a trough arranged in the outer face of the first arm and having a bottom, the through orifice being arranged through the bottom of the trough.

Such a housing for accommodating the optic facilitates the precise positioning and attachment thereof.

In a specific embodiment of the invention, the attachment means comprise at least one magnet and/or a magnetized surface capable of cooperating with a magnet disposed on the optic.

Attachment means with a magnet allow removable attachment of the optic to the sample holder, while remaining robust and easy to use.

In a specific embodiment of the invention, the sample holder is symmetrical with respect to a median longitudinal plane of the sample holder.

Such a symmetry guarantees use of the sample holder suitable for both left-handed users and right-handed users.

The invention also relates to a hair strand analysis kit comprising a sample holder as described above and a hair strand analysis device comprising an optic capable of being removably attached to the sample holder.

In a specific embodiment of the invention, the analysis device is capable of analyzing at least one parameter of a hair strand received in the receiving housing of the sample holder, the analyzed parameter being selected from the hair color, the gray hair percentage, the hair diameter, the hair density, the physical or chemical hair damage status, the hair porosity, fiber pollution.

An analysis of such parameters allows a precise determination of the hair products suitable for the user.

The invention also relates to a hair strand analysis method, comprising the following steps:
providing a kit as described above, the analysis device being at a distance from the sample holder;
selecting a hair strand to be analyzed;
disposing at least a part of the hair strand in the housing for receiving the hair strand; then,
attaching the optic of the analysis device to the sample holder using the attachment means; and
analyzing the hair strand.

The method for dispensing cosmetic product according to the invention can comprise one or more of the following features, taken alone or in any technical possible combination:
The sample holder comprises a first arm and a second arm movable onto one another, each arm comprising an inner face and an outer face, the step of disposing at least a part of the hair strand in the receiving housing comprising disposing the hair strand between the inner faces of the arms.
The hair strand comprises a plurality of hairs, when the part of the hair strand is received in the receiving housing, a part of each hair being housed in the receiving housing, said parts of the hairs being stretched and aligned substantially parallel with one another in the receiving housing, facing the through orifice.
The step of attaching the optic of the analysis device to the sample holder is carried out by the cooperation of at least one magnet disposed on one of the sample holder and the optic and of a magnetized surface on the other of the sample holder and the optic.

The invention will be easier to understand after reading the following description, provided solely as an example, and with reference to the appended drawings, wherein:

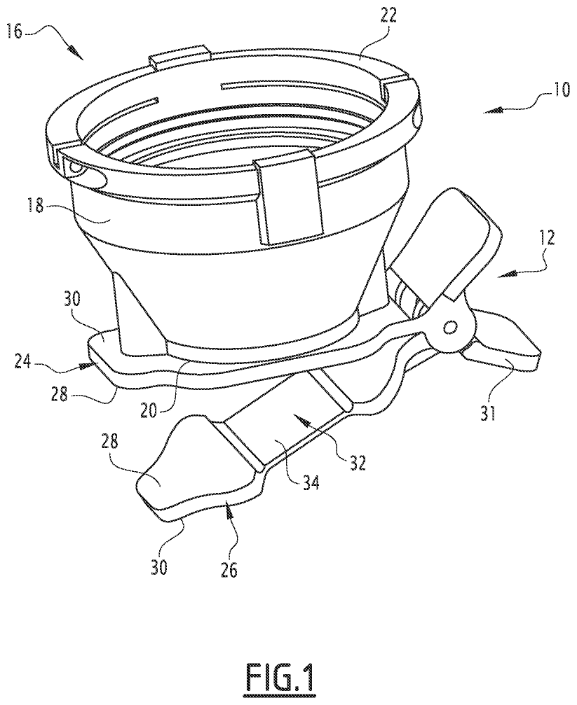

FIG. 1 is a perspective view of a hair strand analysis kit according to the invention;

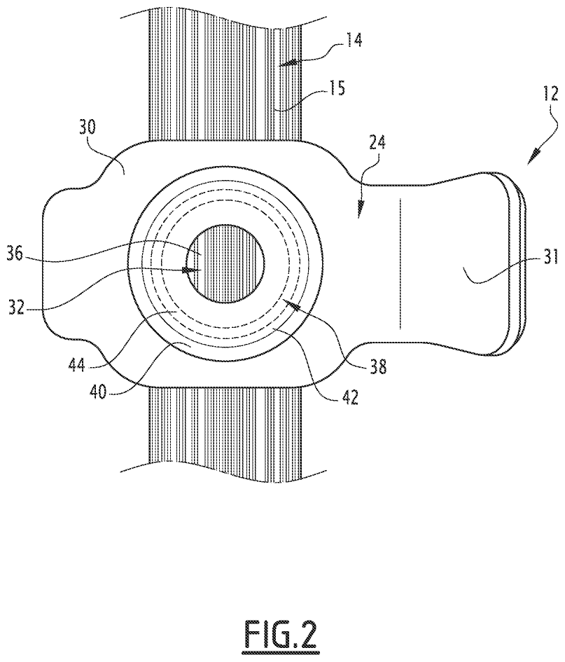

FIG. 2 is a top view of the sample holder of the hair strand analysis kit in FIG. 1 clamping a hair strand;

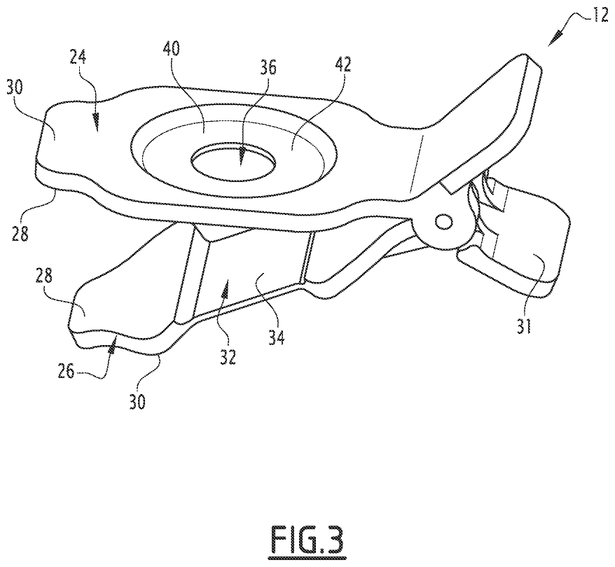

FIG. 3 is a perspective view of the sample holder in FIG. 2; and

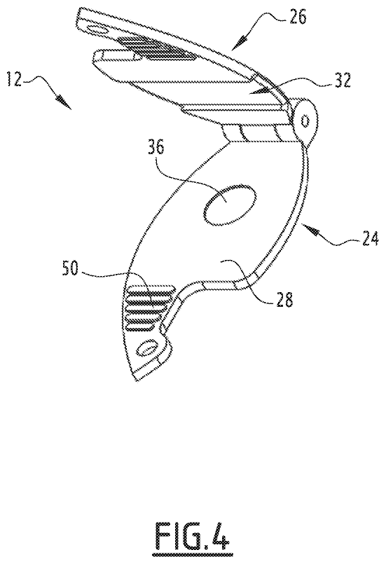

FIGS. 4 to 6 are perspective views of sample holders according to further embodiments of the invention.

FIG. 1 illustrates a hair strand analysis kit 10 comprising a sample holder 12 for supporting a hair strand 14 and a hair strand analysis device 16.

As illustrated in FIG. 2, the hair strand 14 comprises a plurality of hairs 15, and for example between 50 and 1000 hairs.

The analysis device 16 comprises an optic 18 capable of being removably attached to the sample holder 12, as described hereinafter.

As illustrated in FIG. 1, the optic 18 is a frustoconical chamber wherein at least one lens, at least one mirror or a combination of both and optionally at least one lighting element such as a light-emitting diode are disposed.

The frustoconical chamber defines a first free edge 20 and a second mounting edge 22 opposite the first free edge 20.

The second mounting edge 22 is, for example, mounted on a casing (not illustrated) of the analysis device 16, intended to be held in a user's hand.

The first free edge 20 is intended to be placed close to and in the direction of the hair strand to be analyzed, and more specifically the hair strand 14 supported by the sample holder 12.

The first free edge 20 defines a capture opening through which an image of the hair strand 14 to be analyzed is captured or at least one parameter of the hair strand to be analyzed is measured directly.

The analysis device 16 is capable of analyzing at least one parameter of the hair strand 14.

The parameter analyzed is selected from the hair color, the gray hair percentage, the hair diameter, the hair density, the physical or chemical hair damage status, the hair porosity, or/and the fiber pollution.

An example of an analysis device is described in the application U.S. Pat. No. 9,316,580.

In an embodiment of the invention illustrated in the figures and described hereinafter, the sample holder 12 is a clamp.

The clamp 12 comprises a first arm 24 and a second arm 26 hinged on one another and capable of moving from an open position to a closed position, the closed position representing an idle position.

Each arm 24, 26 comprises an inner face 28 and an outer face 30.

The inner faces 28 of the two arms 24, 26 are capable of coming against one another thus defining the closed position of the clamp 12.

In the open position of the clamp 12 as illustrated in FIG. 3, the two inner faces 28 of the two arms 24, 26 are separated from one another.

More specifically, the two arms 24, 26 are connected to one another by a pivot link, and for example by a return spring to the closed position also acting as a hinge at one of the longitudinal ends 31 of the arms 24, 26 of the clamp 12 each defining a support strip.

Using the longitudinal ends 31, particularly by pressing on each longitudinal end 31 to move the longitudinal ends 31 closer and place the strips of each arm 24, 26 against one another, the clamp 12 is moved to the open position.

In the absence of external use, the clamp 12 automatically returns to the closed position

In the closed position, the inner faces 28 of the two arms 24, 26 define therebetween a housing 32 for receiving the hair strand 14.

Holding the clamp 12 in the closed position allows clamping of the hair strand 14 which is received in the receiving housing 32 and therefore a hold in position thereof.

Each arm 24, 26 of the clamp 12 has for example a length between 3 cm and 8 cm and a width between 2 cm and 4 cm.

Advantageously, the inner face 28 of the second arm 26 defines a groove 34 delimiting the receiving housing 32.

The groove 34 extends transversely in relation to a longitudinal axis of the second arm 26. It extends across the entire width of the inner face 28 of the second arm 26 and along a part of the length thereof and delimits a receiving housing 32 of substantially rectangular shape.

The groove 34 defines a bottom of substantially rectangular contour and defining a surface for supporting the hairs 15 of the hair strand 14 received in the groove 34.

The bottom is advantageously a monochrome surface, for example black or white.

Alternatively, the bottom is a transparent surface, a reflective surface or includes patterns.

Such a receiving housing 32 has for example a width between 0.5 cm and 4 cm, and a length between 2 cm and 4 cm.

The groove 34 has for example a depth between 0.5 mm and 3 mm.

The receiving housing 32 thus delimits the width and thickness of the hair strand 14 to be analyzed.

Preferably, as illustrated in FIG. 1, the second arm 26 comprises lateral bulges defined close to the groove 34, on either side of the open ends of the groove 34. The width of the second arm 26 at said bulges is greater than the width of the second arm 26 at the groove 34.

Advantageously, the groove 34 also comprises combing teeth, preferably arranged on the transverse edges of the groove 34. Such combing teeth are capable of holding hairs 15 positioned therein. More specifically, the combing teeth make it possible to keep the hairs 15 stretched and aligned, preferably along a transverse axis of the second arm 26.

The first arm 24 comprises a through orifice 36 and attachment means 38 for removably attaching the optic 18 to the clamp 12.

As illustrated in FIGS. 2 and 3, the through orifice 36 is arranged facing the housing 32 for receiving the hair strand 14, and more specifically is defined facing the groove 34 of the second arm 26.

The through orifice 36 is for example of circular shape, preferably of diameter less than the width of the receiving housing 32, and for example of diameter between 0.5 cm and 3 cm.

The through orifice 36 opens facing the groove 34. As illustrated in FIG. 2, when the clamp 12 is in the closed position, the through orifice 36 extends facing the receiving housing 32 and defines a capture opening through which the hairs 15 of the hair strand 14 can be observed.

The through orifice 36 is intended to removably receive the optic 18 of the analysis device 16.

More specifically, the optic 18 of the analysis device 16 is intended to be received in the through orifice 36, such that the capture opening defined on the first free edge 20 of the optic 18 is aligned with the capture opening defined by the through orifice 36.

Except for the through orifice 36, the first arm 24 is solid, particularly in and around the receiving housing 32.

In order to simplify the positioning of the optic 18, the outer face 30 of the first arm 24 comprises a housing 40 for accommodating the optic of the analysis device arranged around the through orifice 36.

As illustrated in FIGS. 2 and 3, the accommodating housing 40 is a trough arranged in the outer face 30 of the first arm 24 and has a bottom 42.

The through orifice 36 is arranged through the bottom 42 of the trough.

The optic 18 of the analysis device 16 is removably attached to the outer face 30 of the first arm 24, and more specifically by the attachment means 38 in the accommodating housing 40. In particular, the removable attachment can be activated or released manually by the user, preferably after positioning the hair strand 14 in the clamp 12.

The attachment means 38 comprise at least one magnet 44, more specifically disposed around the through orifice 36 in the bottom 42 of the trough.

As illustrated in FIG. 2, the magnet 44 is for example of circular shape surrounding the through orifice 36.

The first free edge 20 of the optic 18 advantageously comprises a magnetized surface capable of cooperating with the magnet 44 so as to attach the optic 18 to the outer face 30 of the first arm 24.

Each arm 24, 26 of the clamp 12 is for example manufactured by injection molding of a plastic material, such as PET.

Advantageously, the clamp 12 is symmetrical with respect to a median longitudinal plane P of the clamp, as illustrated in FIG. 1.

Such a symmetry guarantees use of the clamp 12 suitable for both left-handed users and right-handed users.

A hair strand analysis method will now be described.

A hair strand analysis kit 10 as described above comprising a clamp 12 and an analysis device 16 is firstly provided.

The analysis device 16 is at a distance from the clamp 12.

In other words, the optic 18 of the analysis device 16 is not attached to the outer face of the clamp 12 and the clamp 12 can be moved and actuated separately from the analysis device 16.

A hair strand 14 to be analyzed is selected.

For this purpose, the hair strand 14 is separated, temporarily, from the rest of the user's hair, for example by holding it between the fingers. The clamp 12 is then moved to the open position, for example by applying a pressure on the longitudinal ends 31 of the arms 24, 26 of the clamp 12.

A part of said hair strand 14 is inserted into the receiving housing 32 and more specifically into the groove 34.

The bulges of the second arm 26 and the combing teeth of the groove 34 facilitate the insertion of the hair strand 14 in said groove 34, and more specifically an insertion and hold of the hairs 15 in a stretched and aligned manner.

The clamp 12 is moved back to the closed position, for example by releasing the pressure applied on the longitudinal ends 31 of the arms 24, 26 of the clamp 12.

The hair strand 14 is thus clamped between the inner faces 28 of the arms 24, 26 of the clamp 12.

More specifically, as illustrated in FIG. 2, a part of the hair strand 14 is clamped in the receiving housing 32.

When the part of the hair strand 14 is clamped in the receiving housing 32, a part of each hair 15 of the hair strand 14 is thus housed in the receiving housing 32. The parts of the hairs 15 are stretched and aligned substantially parallel with another in the receiving housing 32 facing the through orifice 36. They extend facing the through orifice 36 so as to be visible through the through orifice 36.

The clamp 12 is thus held in position on the user's hair, while clamping the hair strand 14 to be analyzed.

The optic 18 of the analysis device 16 is then removably attached to the clamp 12 using attachment means 38.

For this purpose, the optic 18 is positioned in the accommodating housing 40 of the clamp 12 held in position on the user's hair.

The optic 18 is then attached to the outer face 30 of the first arm 24 of the clamp 12 by cooperation of the magnet(s) 44 disposed on the clamp 12, and more specifically on the bottom 42 of the trough and of a magnetized surface on the optic 18, and more specifically on the first free edge 20 of the optic 18.

The optic 18 of the analysis device 16 is thus positioned, such that the capture opening defined on the first free edge 20 of the optic 18 is aligned with the capture opening defined by the through orifice 36.

The hair strand 14 is then analyzed, more specifically by capturing an image of the hair strand through the capture opening defined by the through orifice 36 or by directly measuring one of the parameters of the hair strand 14 through the through orifice 36.

The parameter analyzed is for example the hair color, the gray hair percentage, the hair diameter, the hair density, the physical or chemical hair damage status, the hair porosity, and/or the fiber pollution.

When the analysis is complete, the analysis device 16 is once again separated from the clamp 12, for example by pulling the optic 18 so as to separate the optic 18 from the outer face 30 of the first arm 24.

The clamp 12 is then moved to the open position, in order to release the hair strand 14.

Such a clamp 12 is easy to position, thanks to the removable attachment of the optic of the analysis device.

Thus, the hair strand 14 to be analyzed is easily selected and clamped in the clamp 12, without the presence of the optic 18 of the analysis device impeding this positioning.

The precision of the measurements of the analysis device 16 is also improved, thanks to precise positioning and a good aligned hold of the hairs 15 of the hair strand 14.

Furthermore, such a clamp 12 is easy to manufacture and economical.

Such a hair strand analysis kit is also easy to use to implement the hair strand analysis method.

Alternative embodiments of the invention will now be described. Only the features differing from the features of the embodiment described above will be described hereinafter.

In an alternative embodiment of the invention, the attachment means 38 comprise several magnets 44 disposed in the bottom 42 of the trough and distributed in a circular manner around the through orifice 36.

Alternatively, the magnet(s) 44 are disposed at a distance from the bottom 42, for example between the inner face 28 and the outer face 30 of the first arm 24, or on the inner face 28 of the first arm 24 facing the bottom 42 of the trough.

In a further alternative embodiment of the invention, the attachment means 38 comprise a magnetized surface, more specifically on the bottom 42 of the trough, capable of cooperating with at least one magnet 44 disposed on the optic 18, and more specifically the free edge 20 of the optic 18.

In this alternative embodiment of the invention, the optic 18 is attached to the clamp 12 by the cooperation of at least one magnet 44 disposed on the optic 18 and a magnetized surface defined on the clamp 12.

According to a further embodiment, the attachment means 38 include means for snap-locking the optic 18 to the outer face 30 of the first arm 24.

In this embodiment, the optic 18 is attached to the clamp 12 by snap-locking the optic 18 to the outer face 30 of the first arm 24 of the clamp 12.

Alternatively according to the invention, the outer face 30 of the first arm 24 is devoid of a housing 40 for accommodating the optic 18.

In an alternative embodiment of the invention illustrated in FIG. 4, the two arms 24, 26 define holding strips 50 at the opposite end thereof to the hinge. The holding strips 50 are advantageously offset in relation to one another, such that, when the clamp 12 is in the closed position, the latter are not in contact and define a notch therebetween.

In a further embodiment of the invention, the sample holder 12 is not a clamp. This embodiment differs from the embodiments described above in that the sample holder 12 comprises the two arms 24, 26 are not hinged with one another, but are connected by a spacer at one of the longitudinal ends 31 thereof.

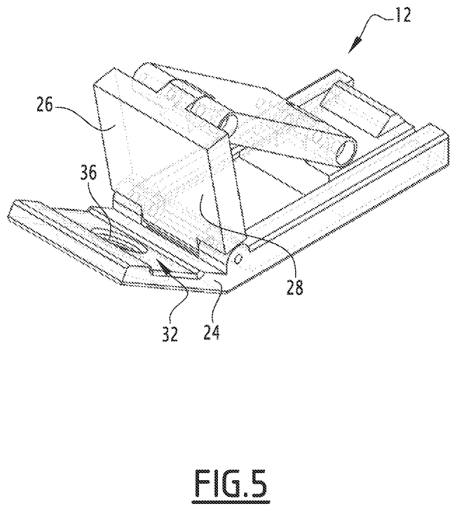

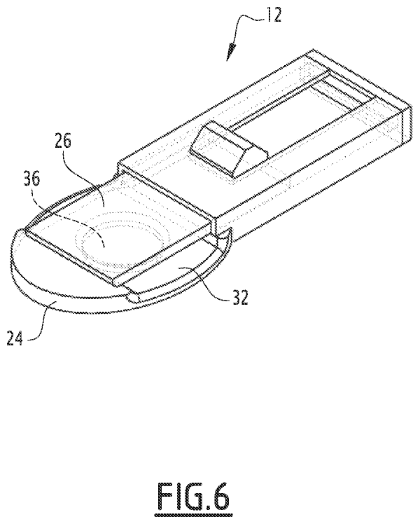

In alternative embodiments of the invention illustrated in FIGS. 5 and 6, the through orifice 36 and the housing 32 for receiving the hair strand 14 are defined on the same arm 24 of the sample holder 12.

In the embodiment illustrated in FIG. 5, the first arm 24 is fixed and the second arm 26 is hinged by an assembly formed from a guillotine and a hinge capable of bringing the inner face 28 of the second arm 26 against the inner face 28 of the first arm 24.

In the embodiment illustrated in FIG. 6, the first arm 24 is fixed and the second arm 26 is a slide capable of moving in translation without rotating to move from an open position to a closed position wherein the second arm 26 covers the receiving housing 32.

Alternatively according to the invention, the sample holder 12 only comprises a first arm 24 and an edge extending perpendicularly to the first arm 24. The receiving housing 32 is defined between the inner face 28 of the first arm 24 and the edge. In such an embodiment, the groove 34 is defined on the inner face 28 of the arm 24. The through orifice 36 is advantageously defined through the bottom of said groove 34.

1. A sample holder for supporting a hair strand, comprising at least a first arm, the first arm comprising an inner face and an outer face, the sample holder defining a housing for receiving the hair strand delimited at least in part by the first arm,
characterized in that the first arm comprises a through orifice arranged facing the housing for receiving the hair strand and intended to removably receive an optic of a hair strand analysis device, the first arm comprising attachment means intended to removably attach the optic of the analysis device to the outer face of the first arm.

2. The sample holder according to claim 1, wherein the sample holder comprises a second arm, each arm comprising an inner face and an outer face, the inner faces of the two arms defining therebetween the housing for receiving the hair strand.

3. The sample holder according to claim 2, wherein the sample holder is a clamp, the first arm and the second arm being hinged on one another, the inner faces of the two arms being capable of coming against one another.

4. The sample holder according to claim 2, wherein the inner face of the second arm defines a groove delimiting the housing for receiving the hair strand supported.

5. The sample holder according to claim 4, wherein the through orifice of the first arm is defined facing the groove.

6. The sample holder according to claim 4, wherein the groove comprises a bottom, the bottom being a monochrome surface, a transparent surface, a reflective surface or including patterns.

7. The sample holder according to claim 4, wherein the second arm comprises bulges defined near the groove.

8. The sample holder according to claim 4, wherein the groove comprises combing teeth.

9. The sample holder according to claim 1, wherein the outer face of the first arm comprises a housing for accommodating the optic of the analysis device arranged around the through orifice.

10. The sample holder according to claim 9, wherein the accommodating housing is a trough arranged in the outer face of the first arm and having a bottom, the through orifice (36) being arranged through the bottom of the trough.

11. The sample holder according to claim 1, wherein the attachment means comprise at least one magnet and/or a magnetized surface capable of cooperating with a magnet disposed on the optic.

12. A hair strand analysis kit comprising:
a sample holder according to claim 1;
a hair strain analysis device comprising an optic capable of being removably attached to the sample holder.

13. The hair according to claim 12, wherein the analysis device is capable of analyzing at least one parameter of a hair strand received in the receiving housing of the sample holder, the analyzed parameter being selected from the hair color, the gray hair percentage, the hair diameter, the hair density, the physical or chemical hair damage status, the hair porosity, fiber pollution.

14. A method for analyzing a hair strand comprising the following steps:
providing a kit according to claim 12, the analysis device being at a distance from the sample holder;
selecting a hair strand to be analyzed;
disposing at least a part of the hair strand in the housing for receiving the hair strand; then,
attaching the optic of the analysis device to the sample holder using the attachment means; and
analyzing the hair strand.

15. The method according to claim 14, wherein the sample holder comprises a first arm and a second arm movable onto one another, each arm comprising an inner face and an outer face, the step of disposing at least a part of the hair strand in the receiving housing comprising disposing the hair strand between the inner faces of the arms.

16. The method according to claim 14, wherein the hair strand comprises a plurality of hairs, when the part of the hair strand is received in the receiving housing, a part of each hair being housed in the receiving housing, said parts of the hairs being stretched and aligned substantially parallel with one another in the receiving housing, facing the through orifice.

17. The method according to claim 14, wherein the step of attaching the optic of the analysis device to the sample holder is carried out by the cooperation of at least one magnet disposed on one of the sample holder and the optic and of a magnetized surface on the other of the sample holder and the optic.

18. The sample older according to claim 3, wherein the inner face of the second arm defines a groove delimiting the housing for receiving the hair strand supported.

19. The sample holder according to claim 5, wherein the groove comprises a bottom, the bottom being a monochrome surface, a transparent surface, a reflective surface or including patterns.

20. The sample holder according to one of claim 5, wherein the second arm comprises bulges defined near the groove.
